(19) United States
(12) Patent Application Publication
(10) Pub. No.: US 2024/0148368 A1
(43) Pub. Date: May 9, 2024

(54) ULTRASOUND DIAGNOSTIC APPARATUS

(71) Applicant: FUJIFILM Healthcare Corporation, Chiba (JP)

(72) Inventor: SOTARO OHARA, Chiba (JP)

(73) Assignee: FUJIFILM Healthcare Corporation, Chiba (JP)

(21) Appl. No.: 18/500,129

(22) Filed: Nov. 2, 2023

(30) Foreign Application Priority Data
Nov. 7, 2022 (JP) 2022-177843

(51) Int. Cl. A61B 8/00

(52) U.S. Cl. CPC A61B 8/565; A61B 8/54

(57) ABSTRACT

A calculator calculates an actual transfer rate of a frame sequence by a communication unit. A determiner determines that frame congestion occurs in a case where the actual transfer rate is lower than a frame rate. A frame rate controller matches the frame rate with the actual transfer rate. As a result, the frame congestion is eliminated. The frame congestion may be eliminated by lowering a load on a processing section.

### CROSS-REFERENCE TO RELATED APPLICATION

This application claims the priority benefits of Japanese application no. 2022-177843, filed on Nov. 7, 2022. The entirety of the above-mentioned patent application is hereby incorporated by reference herein and made a part of this specification.

### BACKGROUND

### 1. Technical Field

The present disclosure relates to an ultrasound diagnostic apparatus, and particularly relates to transferring a frame sequence to an external apparatus.

### 2. Description of the Related Art

In a transmission and reception section of the ultrasound diagnostic apparatus, a frame sequence (frame data sequence) is generated according to a frame rate. Various kinds of processing are applied to the generated frame sequence, and then the processed frame sequence is displayed as a video image.

As necessary, the frame sequence is transferred from the ultrasound diagnostic apparatus to the external apparatus. For example, in an operating room, the frame sequence is transferred from an ultrasound diagnostic apparatus to an information processing apparatus (including other medical apparatuses), the frame sequence is transferred from an ultrasound diagnostic apparatus in an examination room to an information processing apparatus in a diagnostic reading room, or the frame sequence is transferred from an ultrasound diagnostic apparatus in one hospital to an information processing apparatus in another hospital.

The information processing apparatus as a transfer destination includes an analysis unit that analyzes the frame sequence, a processing unit that processes the frame sequence, and the like. In some cases, the information processing apparatus as the transfer destination is required to acquire the entire frame sequence generated by the ultrasound diagnostic apparatus in real time without omission. Hereinafter, such a request will be referred to as a “total acquisition request” in some cases.

In a case where a communication condition between the ultrasound diagnostic apparatus and the external apparatus deteriorates, the entire frame sequence cannot be sent out from the ultrasound diagnostic apparatus to the external apparatus in real time. Specifically, frame congestion occurs during the transfer of the frame sequence, which causes accumulation of transfer delays and frame loss. In this case, the above-mentioned total acquisition request cannot be satisfied.

On the other hand, in the ultrasound diagnostic apparatus, a processor such as a CPU executes a great deal of processing. In general, as the frame rate in the transmission and reception section increases, a load on the processor increases. In a case where the load on the processor is remarkably increased, the frame congestion occurs in the middle of processing the frame sequence. That is, the accumulation of transfer delays and the frame loss occur. In this case as well, the above-mentioned total acquisition request cannot be satisfied.

JP2021-106871A discloses that, in a case where communication is performed between two devices constituting an ultrasound diagnostic system, data to be transferred is switched according to a communication condition between the devices. JP2004-267301A and JP2005-58576A disclose that, in a case where communication is performed between two devices constituting an ultrasound diagnostic system, a data compression rate is changed according to a transmission state between the devices. JP2021-106871A, JP2004-267301A, and JP2005-58576A do not disclose a technique for monitoring transfer of a frame sequence to an external apparatus in an ultrasound diagnostic apparatus.

### SUMMARY

An object of the present disclosure is to transfer a frame sequence from an ultrasound diagnostic apparatus to an external apparatus while avoiding occurrence of frame congestion in the ultrasound diagnostic apparatus. Alternatively, an object of the present disclosure is to prevent accumulation of transfer delays and frame loss during transfer of a frame sequence from an ultrasound diagnostic apparatus to an external apparatus. Alternatively, an object of the present disclosure is to transfer the entire frame sequence generated by transmission and reception of ultrasonic waves from an ultrasound diagnostic apparatus to an external apparatus in real time.

An ultrasound diagnostic apparatus according to the present disclosure comprises: a transmission and reception section that outputs a frame sequence; a processing section that processes the frame sequence output from the transmission and reception section; a communication unit that is provided downstream of the processing section and transfers the frame sequence processed by the processing section to an external apparatus; a monitoring unit that determines whether or not frame congestion occurs in the ultrasound diagnostic apparatus by monitoring the transfer of the frame sequence by the communication unit; and a control unit that reduces a data amount in the frame sequence to be transmitted to the external apparatus by changing at least one of a transmission and reception condition in the transmission and reception section or a processing condition in the processing section in a case where it is determined that the frame congestion occurs.

According to the present disclosure, it is possible to transfer the frame sequence from the ultrasound diagnostic apparatus to the external apparatus while avoiding the occurrence of the frame congestion in the ultrasound diagnostic apparatus. Alternatively, according to the present disclosure, it is possible to eliminate or reduce the accumulation of transfer delays and the frame loss during the transfer of the frame sequence from the ultrasound diagnostic apparatus to the external apparatus. Alternatively, according to the present disclosure, it is possible to transfer the entire frame sequence generated by transmission and reception of ultrasonic waves from the ultrasound diagnostic apparatus to the external apparatus in real time.

### BRIEF DESCRIPTION OF THE DRAWINGS

FIG. 1 is a block diagram showing a configuration example of an ultrasound diagnostic apparatus according to an embodiment.

FIG. 2 is a diagram showing a configuration example of an operation control section.

FIG. 3 is a diagram showing a plurality of restriction modes.

FIG. 4 is a diagram showing a plurality of trial conditions.

FIG. 5 is a flowchart showing a first operation example.

FIG. 6 is a flowchart showing a second operation example.

### DESCRIPTION OF THE EMBODIMENTS

Hereinafter, an embodiment will be described with reference to the drawings.

### (1) Outline of Embodiment

An ultrasound diagnostic apparatus according to the embodiment includes a transmission and reception section, a processing section, a communication unit, a monitoring unit, and a control unit. The transmission and reception section outputs a frame sequence. The processing section processes the frame sequence output from the transmission and reception section. The communication unit is provided downstream of the processing section, and transfers the frame sequence processed by the processing section to an external apparatus. The monitoring unit determines whether or not frame congestion occurs in the ultrasound diagnostic apparatus by monitoring the transfer of the frame sequence by the communication unit. The control unit reduces a data amount in the frame sequence to be transmitted to the external apparatus by changing at least one of a transmission and reception condition in the transmission and reception section or a processing condition in the processing section in a case where it is determined that the frame congestion occurs.

With the above configuration, in a case where it is determined that the frame congestion occurs in the ultrasound diagnostic apparatus, the data amount in the frame sequence to be transmitted to the external apparatus is reduced. As a result, the frame congestion is eliminated or relaxed. In a case where the frame congestion is eliminated, it is possible to transfer the entire frame sequence generated in the ultrasound diagnostic apparatus to the external apparatus in real time. The determination of the occurrence of the frame congestion may include determination of occurrence of a state immediately before the frame congestion.

In the embodiment, a handshake (which can also be called a connection-oriented protocol) is adopted in the transfer of individual frames from the communication unit to the external apparatus. That is, start of transfer of an (n+1)-th frame is allowed after completion of transfer of an n-th frame is confirmed. Under the handshake method, the occurrence of the frame congestion is determined by the monitoring unit.

In the embodiment, the monitoring unit includes a calculator and a determiner. The calculator calculates an actual transfer rate for the frame sequence transferred by the communication unit. The determiner determines that frame congestion occurs in a case where the actual transfer rate is lower than a frame rate in the transmission and reception section. For example, the actual transfer rate is calculated based on the number of transfer frames per unit time, an average transfer time of one frame, or the like.

In the embodiment, the control unit reduces the data amount in the frame sequence to be transferred by the communication unit so that the frame congestion is eliminated. In the embodiment, the transmission and reception condition in the transmission and reception section is a frame rate. The control unit reduces the frame rate in the transmission and reception section in a case where it is determined that the frame congestion occurs. Alternatively, the control unit reduces a data amount in each frame constituting the frame sequence transferred by the communication unit by changing the processing condition in the processing section in a case where it is determined that the frame congestion occurs. For example, the data amount in each frame is reduced by lowering a resolution of each frame.

Examples of the cause of the frame congestion include deterioration of a communication condition between the ultrasound diagnostic apparatus and the external apparatus, and an increase in a processing load in the ultrasound diagnostic apparatus. It is desired to restrict the data amount on an upstream side of a position where the cause of the frame congestion exists. For example, in order to cope with the deterioration of the communication condition, the frame rate in the transmission and reception section may be reduced, or the processing condition in the processing section may be changed to reduce the data amount output from the processing section. In a case where an increase in the load on the processing section is the cause of the frame congestion, the frame rate in the transmission and reception section need only be reduced, or the processing condition in the processing section need only be changed to reduce the load. In a case where the position where the cause of the frame congestion exists cannot be specified, it is desired to reduce the data amount in a reception unit located on the most upstream side. That is, it is desired to reduce the frame rate in the transmission and reception section.

A reception frame rate and a transmission frame rate usually match, but, in a case where a plurality of reception frame data are generated per one electronic scan of a transmission beam, the reception frame rate and the transmission frame rate do not match. In controlling the data amount, it is desirable to pay attention to the reception frame rate.

In the embodiment, the control unit attempts recovery of the data amount in the frame sequence after reducing the data amount in the frame sequence. Specifically, the control unit attempts the recovery of the data amount in the frame sequence in a case where a predetermined trial condition is satisfied. With this configuration, in a case where the condition is improved, the transfer data amount per unit time can be restored to the original amount.

### (2) Details of Embodiment

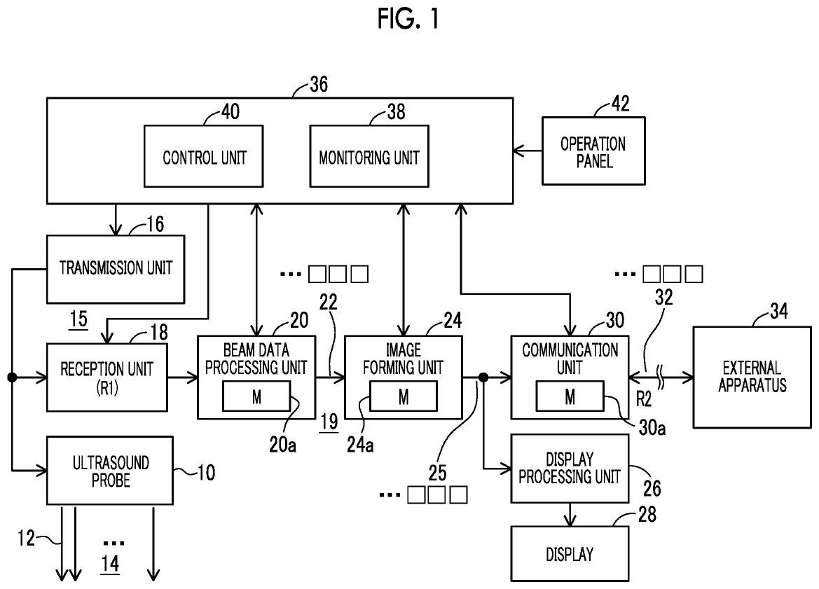

FIG. 1 shows the ultrasound diagnostic apparatus according to the embodiment. This ultrasound diagnostic apparatus is a medical apparatus that is installed in a medical institution such as a hospital and that is used in an ultrasound examination of a subject. The ultrasound diagnostic apparatus is connected to an external apparatus 34 via a network. The external apparatus 34 will be described below.

An ultrasound probe 10 is a device that transmits ultrasonic waves into a living body and receives reflected waves from the living body. The ultrasound probe 10 has a transducer array including a plurality of transducers. An ultrasonic beam 12 is formed by the transducer array, and electronic scanning is performed with the ultrasonic beam 12. As a result, a beam scanning surface 14 is formed. The beam scanning surface 14 is repeatedly formed by repeating the electronic scanning with the ultrasonic beam 12 according to the transmission frame rate.

As the electronic scanning method of the ultrasonic beam 12, an electronic linear scanning method, an electronic sector scanning method, and the like are known. A two-dimensional transducer array may be provided as the transducer array. Volume data can be acquired from a three-dimensional space in the living body by performing two-dimensional scanning with an ultrasonic beam by the two-dimensional transducer array.

A transmission unit 16 is an electronic circuit that functions as a transmission beam former, and outputs a plurality of transmission signals to the transducer array in parallel in a case of performing transmission. Accordingly, the transmission beam is formed by the action of the transducer array.

A reception unit 18 is an electronic circuit that functions as a reception beam former, and applies phasing addition to a plurality of reception signals output in parallel from the transducer array in a case of performing reception, thereby generating reception beam data. A plurality of reception frames (a plurality of reception frame data) are sequentially output from the reception unit 18 with the repetition of the electronic scanning. Specifically, the plurality of reception frames are sequentially output according to a frame rate R1.

Each reception frame is formed of a plurality of reception beam data arranged in an electronic scanning direction. Each reception beam data is formed of a plurality of echo data arranged in a depth direction. The plurality of reception frames output from the reception unit 18 constitute a reception frame sequence. The transmission unit 16 and the reception unit 18 constitute the transmission and reception section 15.

A beam data processing unit 20 is a module that applies a plurality of kinds of processing to each reception beam data. The plurality of kinds of processing may include logarithmic transformation, filtering, and the like. Such kinds of processing may be realized by a function of the CPU. A reception frame sequence 22 output from the beam data processing unit 20 is input to an image forming unit 24. In a configuration example shown in the drawing, the beam data processing unit 20 has a buffer memory 20a that temporarily stores a reception frame sequence before or after processing. The buffer memory 20a has, for example, a ring buffer structure.

The image forming unit 24 has a digital scan converter (DSC) and an image processing unit. The DSC has a coordinate transformation function. The coordinate transformation function is a function of performing transformation from a transmission and reception coordinate system to a display coordinate system. The DSC generates a display frame sequence from the reception frame sequence. In the embodiment, frame rate transformation is not executed in the DSC. Note that the frame rate transformation may be executed.

For example, the display frame sequence constitutes a B-mode tomographic image as a video image. The display frame sequence may constitute volume data as a video image. The image processing unit in the image forming unit 24 applies necessary image processing to the generated display frame sequence.

The image forming unit 24 has a buffer memory 24a that temporarily stores the reception frame sequence or the display frame sequence. The buffer memory 24a has, for example, a ring buffer structure. A display frame sequence 25 output from the image forming unit 24 is sent to the display processing unit 26 and the communication unit 30. The beam data processing unit 20 and the image forming unit 24 constitute a processing section 19.

The display processing unit 26 has an image synthesis function, a rendering function, and the like. A display 28 is connected to the display processing unit 26. The display frame sequence is displayed on a screen of the display 28.

The communication unit 30 transfers a display frame sequence (hereinafter, simply referred to as a frame sequence) 32 to be transferred, to the external apparatus 34, as necessary. The external apparatus 34 is, for example, a computer (or another medical apparatus) installed in an operating room together with the ultrasound diagnostic apparatus, a computer installed in a diagnostic reading room in the same hospital, or a computer installed in another hospital.

The communication unit 30 is connected to the external apparatus 34 via the network. The network includes a wireless LAN, a wired LAN, and the like. A handshake is executed in a case where the communication unit 30 communicates with the external apparatus 34. That is, data is exchanged according to the connection-oriented protocol. An example of such a protocol is TCP/IP. After completion of transfer of an n-th frame to the external apparatus 34 is confirmed in the communication unit 30, transfer of an (n+1)-th frame from the communication unit 30 to the external apparatus 34 is started. This process is repeated. R2 indicates an actual transfer rate of the frame sequence. The communication unit 30 has a buffer memory 30a that temporarily stores a frame sequence before transfer. The buffer memory 30a has a ring buffer structure.

Each of the beam data processing unit 20, the image forming unit 24, the communication unit 30, and the display processing unit 26 can be configured by a processor or the like. These functions may be realized by the CPU described below.

An operation control section 36 is configured by the CPU that executes a program. The operation control section 36 controls an operation of each component in the ultrasound diagnostic apparatus. An operation panel 42 is connected to the operation control section 36. In FIG. 1, two typical functions of the operation control section 36 are represented by two blocks. Specifically, the operation control section 36 functions as a monitoring unit 38 and a control unit (data amount control unit) 40.

The monitoring unit 38 monitors the transfer of the frame sequence by the communication unit 30. As will be described below, the monitoring unit 38 calculates the actual transfer rate (the number of transfer frames per unit time) R2. The monitoring unit 38 determines whether or not the frame congestion (which can also be called a transfer delay) occurs based on the actual transfer rate R2.

The control unit 40 reduces the frame rate and/or changes the processing condition of the processing section such that the data amount in the frame sequence to be transferred is reduced in a case where it is determined that the frame congestion occurs. The control of reducing the data amount is continued until the frame congestion is eliminated.

In a case where a communication condition of the network deteriorates, the actual transfer rate R2 of the frame sequence 32 decreases, and as a result, the number of frames stored in the buffer memory 30a increases. On the other hand, in a case where a load on the processing section 19 increases, a processing rate in the processing section 19 decreases, and as a result, the number of reception frames or the number of display frames stored in the buffer memory 10a or the buffer memory 24a increases. Under the influence of this, the actual transfer rate R2 of the frame sequence also decreases.

In the above two cases, the entire frame sequence (reception frame sequence) generated in the transmission and reception section 15 cannot be sent to the external apparatus 34 in real time. That is, the accumulation of transfer delays and the frame loss occur in the ultrasound diagnostic apparatus. In other words, the full frame transfer request cannot be satisfied.

According to the above-described embodiment, the data amount in the frame sequence to be transferred is reduced until the frame congestion in the ultrasound diagnostic apparatus is eliminated. Therefore, it is possible to transfer the entire frame sequence generated in the ultrasound diagnostic apparatus to the external apparatus in real time.

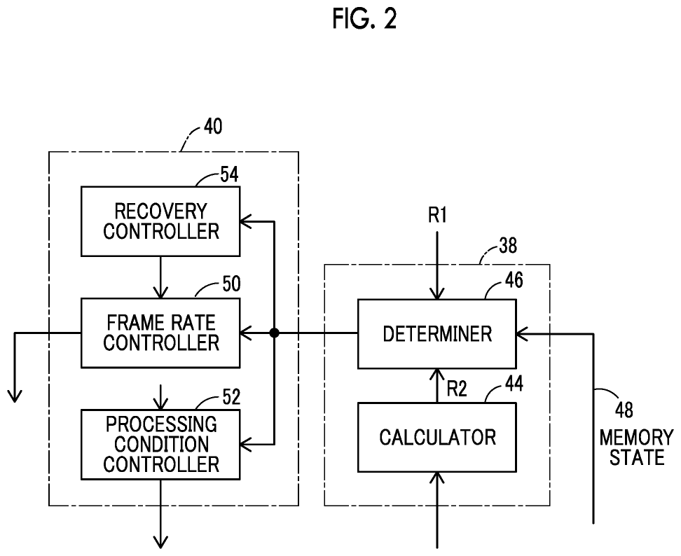

FIG. 2 shows a configuration example of the monitoring unit 38 and the control unit 40. The monitoring unit 38 has a calculator 44 and a determiner 46. The calculator 44 calculates the actual transfer rate R2 by specifying the number of frames to be transferred per unit time or by specifying an average time required for the transfer of one frame. For example, the actual transfer rate R2 can be calculated by obtaining a reciprocal of the average time.

The determiner 46 determines that frame congestion occurs in a case where the actual transfer rate R2 is lower than the frame rate R1. The number of frames temporarily stored in a buffer memory in the monitoring unit or an increase thereof may be specified, thereby determining whether or not the frame congestion occurs.

The control unit 40 has a frame rate controller 50 and a processing condition controller 52, and further has a recovery controller 54. In the embodiment, the frame rate controller 50 and/or the processing condition controller 52 operates according to a restriction mode selected by a user from a plurality of restriction modes.

In a case where it is determined that the frame congestion occurs, the frame rate controller 50 executes control of lowering the frame rate until the frame congestion is eliminated. The frame rate controller 50 may match the frame rate R1 with the actual transfer rate R2. In a case where it is determined that the frame congestion occurs, the processing condition controller 52 changes the processing condition in the processing section, and executes control of reducing the load on the processing section until the frame congestion is eliminated.

The recovery controller 54 trially executes recovery control of increasing the data amount in the frame sequence to be transferred in a case where a predetermined trial condition is satisfied. However, in a case where the frame congestion occurs again, the execution of the recovery control is restricted.

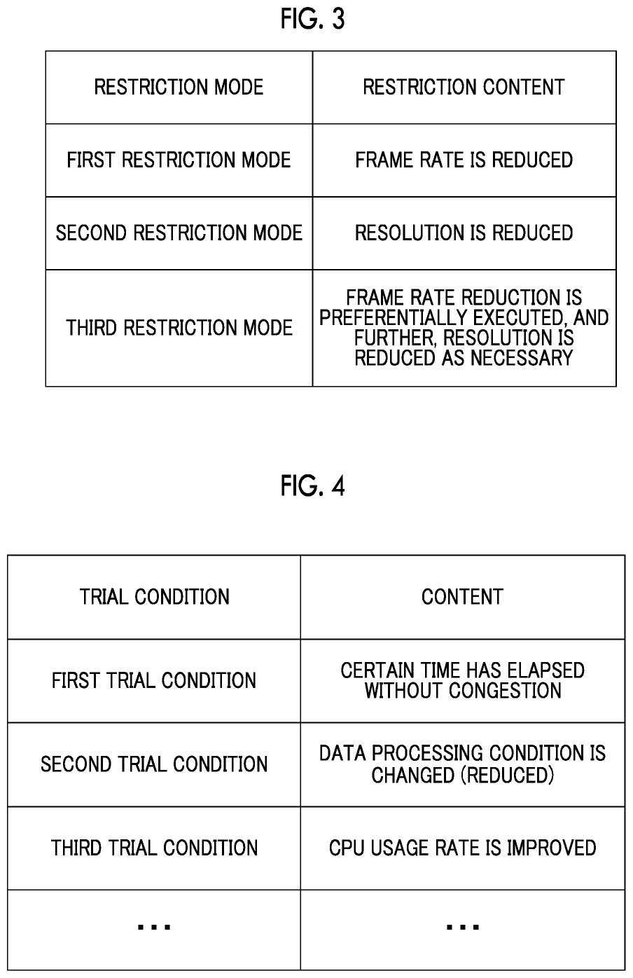

FIG. 3 shows a first restriction mode, a second restriction mode, and a third restriction mode. In a case where the first restriction mode is selected, only the frame rate controller functions at a time when it is determined that the frame congestion occurs, that is, only the reduction of the frame rate is executed. In a case where the second restriction mode is selected, only the processing condition controller functions at a time when it is determined that the frame congestion occurs, that is, only the change of the processing condition of the processing section is executed. In a case where the third restriction mode is selected, at a time when it is determined that the frame congestion occurs, first, the control of reducing the frame rate is preferentially executed, and, in a case where the frame congestion cannot be eliminated even by the control, the processing condition is changed to reduce the data amount in each frame. Specifically, a resolution of each frame is lowered.

Under a condition where a location of the cause of the frame congestion cannot be specified, it is desirable to select the first restriction mode. In a case where the data amount in the frame sequence is reduced in the reception unit located on the most upstream side, the frame congestion can be eliminated regardless of the location of the cause of the frame congestion. In a case where it is clear from information such as a CPU usage rate that the cause of the frame congestion exists in the processing section, it is desired to select the second restriction mode.

FIG. 4 shows a first trial condition, a second trial condition, and a third trial condition. Each trial condition is a condition for activating the recovery control. The first trial condition is a condition that is satisfied in a case where an elapsed time after the frame congestion is eliminated reaches a certain time. The second trial condition is a condition that is satisfied in a case where the change of the data processing condition reduces a burden on the processing section. The third trial condition is a condition that is satisfied in a case where the CPU usage rate drops to a certain value. The user selects one of the trial conditions.

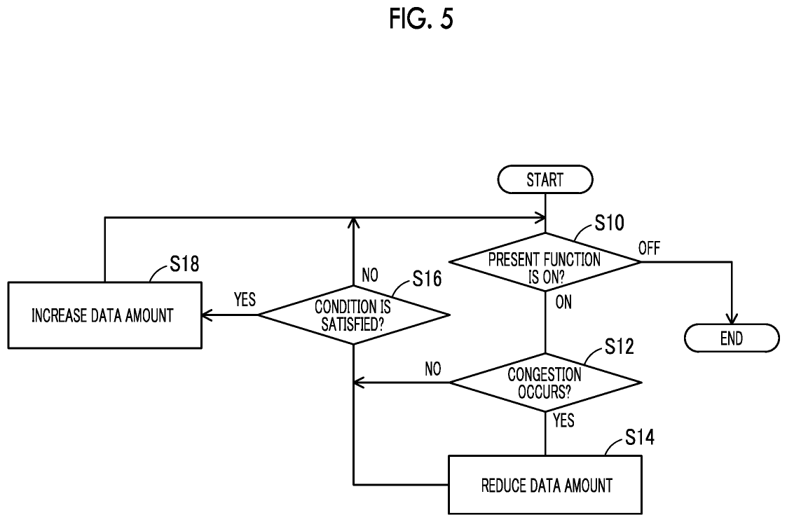

FIG. 5 shows a first operation example of the ultrasound diagnostic apparatus according to the embodiment. In S10, it is determined whether the function (present function) for eliminating the frame congestion is ON or OFF. In a case where it is determined to be ON in S10, it is determined in S12 whether or not the frame congestion occurs. In a case where the frame congestion occurs, the data amount in the frame sequence to be transferred is reduced in S14. The method is decided by the restriction mode selected by the user. In S16, it is determined whether or not the trial condition selected by the user is satisfied. In a case where the trial condition is not satisfied, each step from S10 is executed again. In a case where it is determined in S16 that the trial condition is satisfied, control of increasing the data amount in the frame sequence by a predetermined amount is trially executed in S18. After that, each step after S10 is executed again. In a case where the frame congestion occurs after the increase in the data amount, the data amount is reduced in S14.

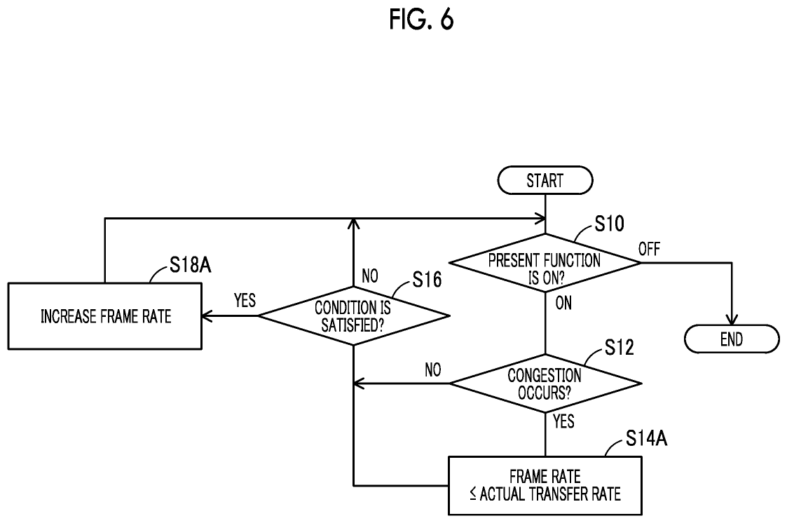

FIG. 6 shows a second operation example of the ultrasound diagnostic apparatus according to the embodiment. In the second operation example, the same steps as those shown in FIG. 5 are denoted by the same step numbers, and the description thereof will be omitted.

In the second operation example, the first restriction mode and the first trial condition are selected. In a case where it is determined in S12 that the frame congestion occurs, the frame rate is changed to be equal to or lower than an actual transfer rate in S14A. In a case where it is determined in S16 that the elapsed time after the elimination of the frame congestion has reached a certain time, the frame rate is increased by a predetermined value in S18A. After that, each step after S10 is executed again.

In the embodiment, a plurality of volume data may be sequentially transferred from the ultrasound diagnostic apparatus to the external apparatus. Each volume data is formed of a plurality of frames that are spatially arranged. The transfer of the plurality of volume data is actually the transfer of the frame sequence. The external apparatus is, for example, an information processing apparatus having an image processing function or an image analysis function. The external apparatus may be another medical apparatus.

What is claimed is:

1. An ultrasound diagnostic apparatus comprising:
a transmission and reception section that outputs a frame sequence;
a processing section that processes the frame sequence output from the transmission and reception section;
a communication unit that is provided downstream of the processing section and transfers the frame sequence processed by the processing section to an external apparatus;
a monitoring unit that determines whether or not frame congestion occurs in the ultrasound diagnostic apparatus by monitoring the transfer of the frame sequence by the communication unit; and
a control unit that reduces a data amount in the frame sequence to be transmitted to the external apparatus by changing at least one of a transmission and reception condition in the transmission and reception section or a processing condition in the processing section in a case where it is determined that the frame congestion occurs.

2. The ultrasound diagnostic apparatus according to claim 1,
wherein the monitoring unit includes
a calculator that calculates an actual transfer rate for the frame sequence transferred by the communication unit, and
a determiner that determines that the frame congestion occurs in a case where the actual transfer rate is lower than a frame rate in the transmission and reception section.

3. The ultrasound diagnostic apparatus according to claim 1,
wherein the control unit reduces the data amount in the frame sequence transferred by the communication unit so that the frame congestion is eliminated.

4. The ultrasound diagnostic apparatus according to claim 1,
wherein the transmission and reception condition in the transmission and reception section is a frame rate, and
the control unit reduces the frame rate in the transmission and reception section in a case where it is determined that the frame congestion occurs.

5. The ultrasound diagnostic apparatus according to claim 1,
wherein the control unit reduces a data amount in each frame constituting the frame sequence transferred by the communication unit by changing the processing condition in the processing section in a case where it is determined that the frame congestion occurs.

6. The ultrasound diagnostic apparatus according to claim 1,
wherein the control unit attempts recovery of the data amount in the frame sequence after reducing the data amount in the frame sequence.

7. The ultrasound diagnostic apparatus according to claim 6,
wherein the control unit attempts the recovery of the data amount in the frame sequence in a case where a predetermined trial condition is satisfied.
